(19) United States
(12) Patent Application Publication
(10) Pub. No.: US 2026/0285864 A1
(43) Pub. Date: Sep. 24, 2026

(54) ULODESINE SALT

(71) Applicant: Laevoroc Immunology AG, Hunenberg (CH)

(72) Inventors: Yao Han, Hunenberg (CH); Xianfeng Chai, Hunenberg (CH); Juan Liu, Hunenberg (CH); Quihong Luo, Hunenberg (CH); Thomas Mehrling, Hunenberg (CH)

(21) Appl. No.: 18/580,646

(22) PCT Filed: Jul. 20, 2022
(86) PCT No.: PCT/EP2022/070369

§ 371 Date: Jan. 19, 2024

(30) Foreign Application Priority Data
Jul. 20, 2021 (GB) 2110403.9

(51) Int. Cl. C07D 487/04; A61K 31/519; C07C 55/12

(52) U.S. Cl. CPC C07D 487/04; A61K 31/519; C07C 55/12

(57) ABSTRACT

The present disclosure relates specifically to novel glutarate salt forms of Ulodesine or the compound known as 7-[(3R, 4R)-3-Hydroxy-4-hydroxymethyl-pyrrolidin-1ylmethyl]-3, 5-dihydro-pyrrolo [3,2-d]pyrimidin-4-one and methods of preparing the same.

### CROSS REFERENCE TO RELATED APPLICATIONS

This application is a U.S. National Stage Application filed under 35 U.S.C. § 371, based on International Patent Application No. PCT/EP2022/070369, filed on Jul. 20, 2022, which claims priority to UK Patent Application No. GB2110403.9, filed on Jul. 20, 2021. The entire contents of each of the above applications are incorporated herein by reference.

The present disclosure relates specifically to novel glutarate salt forms of ulodesine or the compound known as 7-[(3R, 4R)-3-Hydroxy-4-hydroxymethyl-pyrrolidin-1-ylmethyl]-3,5-dihydro-pyrrolo [3,2-d]pyrimidin-4-one and methods of preparing the same.

### BACKGROUND

Ulodesine or the pharmaceutical compound known as 7-[(3R,4R)-3-Hydroxy-4-hydroxymethyl-pyrrolidin-1-ylmethyl]-3,5-dihydro-pyrrolo [3,2-d]pyrimidin-4-one, structurally known as:

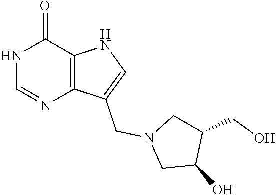 [SMILES: O=c1[nH]cnc2c(CN3C[C@H](CO)[C@@H](O)C3)c[nH]c12]

inhibits a number of relevant enzymes implicated in human disease, including purine nucleoside phosphorylase (PNP).

Ulodesine has been developed for the treatment of a number of human disease, including, but not limited to gout, skin disorders, cancer, B and T-cell mediated disease, bacterial infections and protozoal infections. Use of Ulodesine is also described, for example, in U.S. Pat. No. 7,553,839.

Furthermore, pharmaceutical salts of compound Ulodesine are well known in the literature.

These include, but are not limited to, hydrochlorides, dihydrochorides, hydrobromides, hemi sulfate, p-tosylate, phosphate, citrate, L-tartrate, L-lactate, stearate, maleate, succinate, fumarate, and L-malate. Furthermore, hemi and mono salts of compound with C4 organic diacids may include succinic acid, fumaric acid, L-malic acid, maleic acid, L-tartaric acid, L-aspartic acid and have been exemplified in the art.

While a number of salts of ulodesine have been described, many of the salt forms display properties that are not optimal for methods of production and/or application in pharmaceuticals. For example, the hydrochloride or other salts have been shown to contain polymorphic variants. It may be desirable to obtain a salt of a pharmaceutical compound with no, or a decreased number of polymorphic variants that is stable in a crystal form.

Mixed salts may also offer the potential for physical properties that are different from those of the non-mixed salts alone and so can be helpful in the manufacturing of drug products, whose suitability for use depends on the properties of the active pharmaceutical ingredient. Like unmixed salts, mixed salts are often polymorphic and some of these are unstable.

Further, as regards processing, there remain considerable technical barriers associated with making a useful ulodesine salt. It is noteworthy that even replicating Ulodesine in free form, as referenced in Org. Process Research and Dev. 2009, 13, 928 is not without challenge itself. Thereafter, producing a stable salt and providing a reliable and consistent method for doing so present a second hurdle. Methods for forming a variety of salts of Ulodesine have been described but to date such methods have not identified or produced a candidate salt for the required use in pharmaceuticals.

Therefore, it is desirable to develop stable salts (whether mixed or non-mixed) and processes for their production that will be useful in the manufacture of an improved ulodesine pharmaceutical.

### SUMMARY OF THE INVENTION

In a first aspect, the presently disclosed invention comprises at least one salt of ulodesine, the salt being selected from a glutarate, a malonate and/or an oxalate salt.

Such salt forms have not previously been found, characterised or made from the prior art, yet the inventors have worked at length to generate suitable additional candidates for clinical use and characterised the relevant selections to determine their suitability for potential medical application. Such work has gone beyond the mere typical work undertaken during routine selection of salts since these salts have been challenging to produce in the first instance.

In embodiments the salt comprises a hemi salt. The hemi salt stoichiometry results in stable forms, which in some cases may convert to an anhydrous form upon heating and drying. However, it was a technical challenge to find a method which resulted in the stable hemi form and this was overcome by the present inventors.

In embodiments, the salt comprises a hemi glutarate and in preferred embodiments, a hemi L-glutarate crystalline salt of ulodesine.

Although, initially technically challenging to produce, it was ultimately found that the hemi L-glutarate crystal form exhibited no polymorphic variants, as compared to other stable salt forms of ulodesine known in the art. A physically stable salt, which does not exhibit polymorphs, is a very desirable property in pharmaceutical manufacturing. Furthermore, the hemi L-glutarate has very good physical stability when compared with other tested salts. Further, this selected salt compared favourably with the pharmaceutically acceptable succinate salt and was shown to be more favourable than the succinate salt, showing surprisingly less degradation over two week period in some conditions.

Previous disclosures have not been useful to ascertain the preparation nor permit the characterisation of certain novel salts of ulodesine as described and belonging to the scope of the invention asserted herein. The art typically uses ulodesine free base as starting material to prepare the corresponding salt. Very little previous work has been done to elucidate whether this particular form would meet criteria that are acceptable for salt selection. The absence of literal direction appears to relate to the challenges of recrystallization, finding a way of making a stable crystal form is inventive since there is nothing in the art to teach or direct how to produce it and the routine methods did not result in a usable crystal form. There is no direction in the prior art that the recrystallization from a specific solvent results in that form or other specific new form which is known to be stable and useful.

The glutarate salt made by the inventors was not a clear, nor an obvious candidate for selection as compared to other salts available. Typically, salt selection requires consideration of safety and can includes review of several analytical parameters to determine other helpful chemical and physical properties such as clear and sharp diffraction peaks in graphics, consideration of any obvious amorphous peaks, solvent weight loss and the ability to obtain a crystal form under a variety of conditions. On initial review, the glutarate salt met the safety considerations, for example, glutaric acid appears acceptable for safety but would otherwise not be an immediate choice of a salt under the standard criteria. For example, initial analysis indicated low crystallinity (no sharp diffraction peak) and some obvious solvent weight loss. However, extensive further experiments and testing were conducted in order to elucidate and determine if the hemi glutarate salt form could form stable crystals, since this was unknown from previous work in the art.

Thereafter, the applicant encountered technical challenges required to be overcome to obtain it in the stable, crystal form. For example, it was required that the hemi glutarate salt is made first, and further re-crystallization with different solvents in different orders and with different conditions was necessary to afford the stable crystal, rather than crystallization being standardized. The inventors were successful in determining a stable crystal form and in embodiments it is disclosed that where the salt is a glutarate salt it maybe (a) from 50% to 100% crystalline, and more particularly at least 50% crystalline, or at least 60% crystalline, or at least 70% crystalline, or at least 80% crystalline, or at least 90% crystalline, or at least 95% crystalline, or at least 98% crystalline, or at least 99% crystalline, or at least 99.5% crystalline, or at least 99.9% crystalline, for example 100% crystalline.

Further, useful stable salt mixes are made plausible and feasible by the studies of the herein, wherein an additional salt is already known to be stable but which may be benefitted from having the new hemi L-glutarate salt included therewith.

Such combinations avoid problems associated with salt mixes of the art, for example relating to instability and polymorphic behaviour. In one embodiment, the invention may therefore comprise a composition comprising at least the glutarate salt (as defined and described herein) in combination with a further second pharmaceutically acceptable, physically and chemically second stable salt. In embodiments, this second salt may include a hemi succinate salt.

In embodiments, the salt of ulodesine may be selected from a differently characterised salt, such as an amorphous hemi salt, such as an oxalate and hemi malonate salt. The inventors have also been able to make and characterise these alternative salts for the first time and in doing so offer a further alternative option for pharmaceutical manufacturing.

In embodiments a combination of two or more of these salts may be selected from a glutarate, hemi glutarate, or hemi L-glutarate, and a malonate or an oxalate salt, for example.

The inventors were able to determine good formulation stability in water for the salt of the invention and thus a useful scale-up possibility for formulating in pharmaceuticals.

This work is believed to be helpful in supporting further work on IV formulations, for example and for use within animal studies and clinical trials. The invention therefore further relates to a pharmaceutical compound comprising a salt form of ulodesine of the invention, or salt composition mix, as hereinbefore described.

The invention also extends to a pharmaceutical compound comprising a therapeutically effective amount of the salt form describe herein, or composition of salt mix, of the invention, as described herein, for use as a medicament. In that instance, the pharmaceutical compound may be for use as an inhibitor of PNP.

In a further aspect, the present disclosure provides methods for preparing a hemi glutarate salt or hemi L-glutarate salt of ulodesine. It has been elucidated that recrystallization steps are very critical to obtain a hemi salt or hemi L-salt, in particular: ethanol in conjunction with other steps is necessary for the formation of hemi glutarate crystal, otherwise only other salts such as mono salts (1:1) are obtained, which is less desirable.

The present disclosure provides methods of preparing an ulodesine hemi glutarate salt or an ulodesine hemi L-glutarate salt comprising the steps of:
(a) preparing a solution of ulodesine free base in water, and optionally stirring at room temperature;
(b) adding glutarate acid, or L-glutarate acid to the mixture of step (a), and optionally stirring for 30 min at room temperature;
(c) freeze-drying the solution of step (b) to yield a white solid product;
(d) dissolving the solid product of (c) in water; optionally heating up to 75° C.; adding ethanol and optionally stirring at 75° C. for 30 minutes, to form a homogeneous solution;
(e) making a dropwise addition of acetonitrile to the solution of (d), optionally over a period of 60 minutes;
(f) stirring the solution of (e) for 60 minutes, optionally at 75° C.; and optionally cooling the solution to 0° C. over a period of 60 minutes;
(h) Filtering and washing with acetonitrile to obtain ulodesine hemi salt glutarate salt.

Still further, in the methods above, when the glutarate acid is added it may be added with an amount of the desired final salt form to aid in the crystallization process.

In some embodiments, the disclosure therefore provides methods of preparing a glutarate salt of ulodesine in particular, a hemi glutarate salt, or hemi L-glutarate salt.

The methods above may include holding times after one or more of the steps disclosed.

In embodiments the method of preparing a ulodesine hemi glutarate salt above may further include that the step (a) requires the use of a free form of reactant:

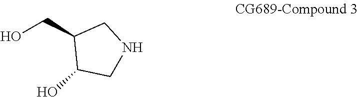

in the preparation of the solution of ulodesine freebase in water.

Desirably, methods according to the invention and embodiments thereof enable both reliable and consistent production of the hemi glutarate salt of ulodesine, which has not be possible from the prior art.

It has been possible by significant experimentation and further technical modification thereof to identify, obtain and increase the yield necessary of this newly described salt for onward pharmaceutical and clinical processing in the desired application. The considerable technical challenges associated with the failure of an identifiable and reproducible new salt candidate have therefore been met, as well as confirming its practical use in formulation and batch production for onward application in medicine and in the treatment of disease.

In embodiments, the salt compounds of the present disclosure can also be prepared according to the specific examples additionally set out below in the description.

### Terms and Abbreviations

As used herein, the following terms and standard methods have the meanings set out below.

The term “API” refers to the active pharmaceutical ingredient.

The term “mono” means the ratio of API: acid is 1:1, respectively, in the crystal structure of the salt of compound Ulodesine.

The term “hemi” means the ratio of API: acid is 2:1, respectively, in the crystal structure of the salt of compound Ulodesine.

The term “inert organic solvent” refers to a solvent that does not interfere chemically with the reaction.

The term “isostructural” is used to describe crystalline substances that have the same type of crystalline structure such as when a new molecular entity substitutes for another in a crystal structure without significantly disturbing the unit cell.

The term “pharmaceutically acceptable,” such as pharmaceutically acceptable carrier, excipient, etc., means pharmacologically acceptable and substantially non-toxic to the subject to which the particular compound is administered.

The term “pharmaceutically acceptable salt” refers to a salt which retains the biological effectiveness and properties of known standard compounds likely to be acceptable for pharmaceutical use and is non-toxic.

### Abbreviations

ACN: Acetonitrile
DSC: Differential scanning calorimeter
DMSO: Dimethyl sulfoxide
DVS: Dynamic vapour sorption
EtOH: Ethanol
FaSSIF: Fasted state simulated intestinal fluid
FeSSIF: Fed state simulated intestinal fluid
HPLC: High performance liquid chromatograph
MeOH: Methanol
NMR: Nuclear magnetic resonance
PLM: Polarized light microscope
RT: Room temperature
RRT: Relative retention time
SGF: Simulated gastric fluid
TFA: Trifluoroacetic acid
TGA: Thermo gravimetric analyser
THF: Tetrahydrofuran
TRS: Total related substance
XRPD: X-ray powder diffraction

### BRIEF DESCRIPTION

The following figures help provide the graphic analysis resulting from various analytical testing showing the physical and chemical properties of the salts in the present disclosure.

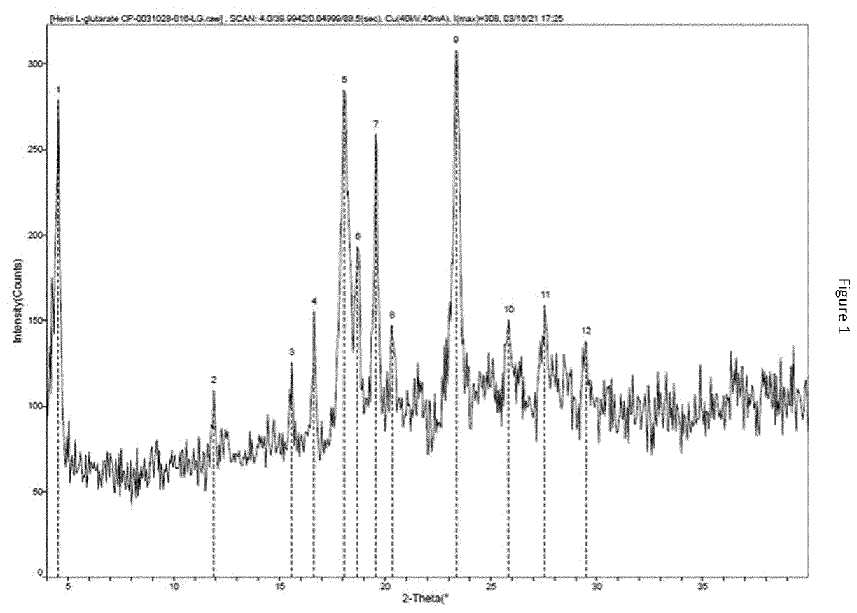

FIG. 1 shows the XRPD labelled pattern of the hemi L-glutarate salt of ulodesine, provided and made in accordance with the present disclosure;

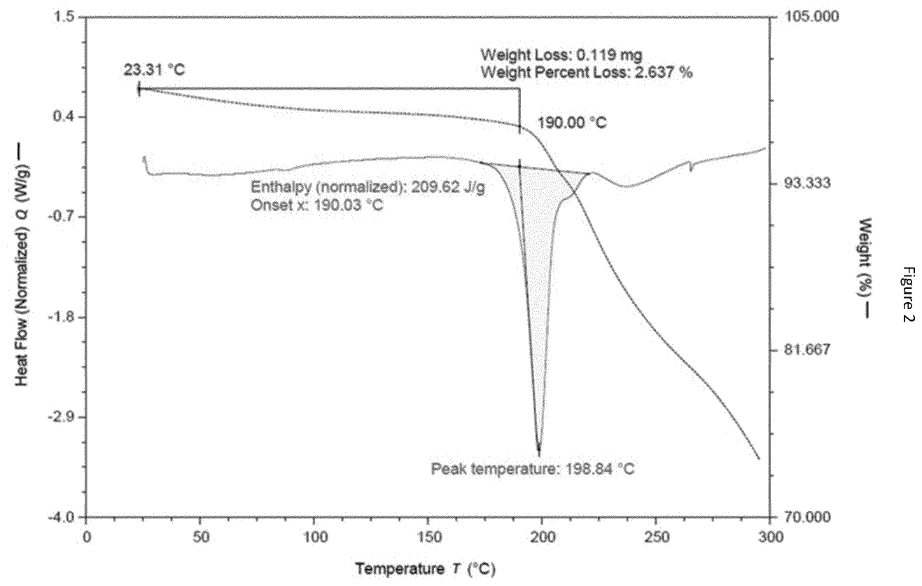

FIG. 2 shows the DSC and TGA overlay of the same hemi L-glutarate salt of ulodesine, provided and made in accordance with the present disclosure;

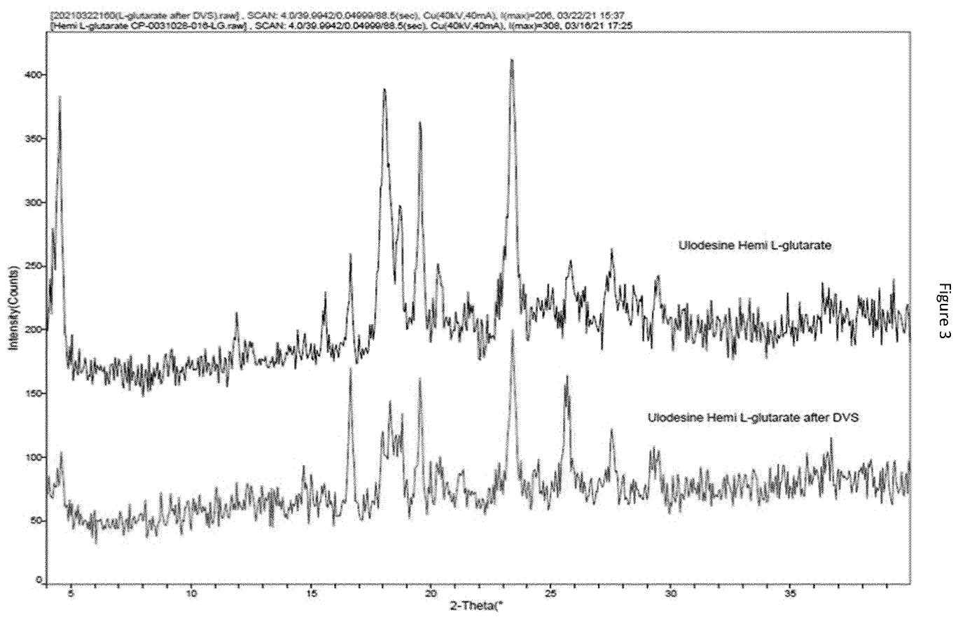

FIG. 3 shows the XRPD overlay of the hemi L-glutarate salt of ulodesine, pre and post DVS, in accordance with the present disclosure;

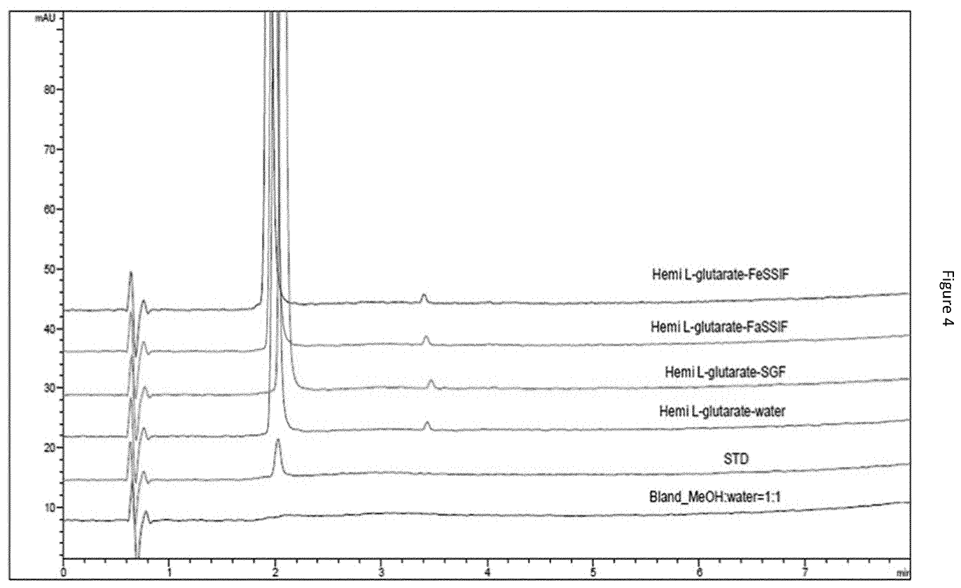

FIG. 4 shows the HPLC overlay demonstrating high solubility of the samples of hemi L-glutarate salt of ulodesine, which has been provided and made in accordance with the present disclosure;

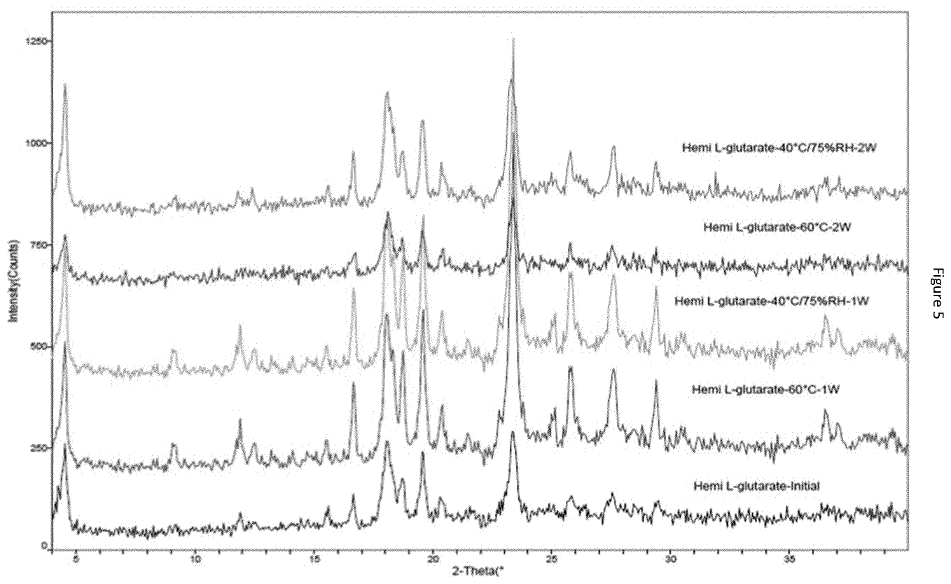

FIG. 5 shows the XRPD overlay of hemi L-glutarate salt provided in accordance with the disclosure herein, demonstrating excellent solid state stability;

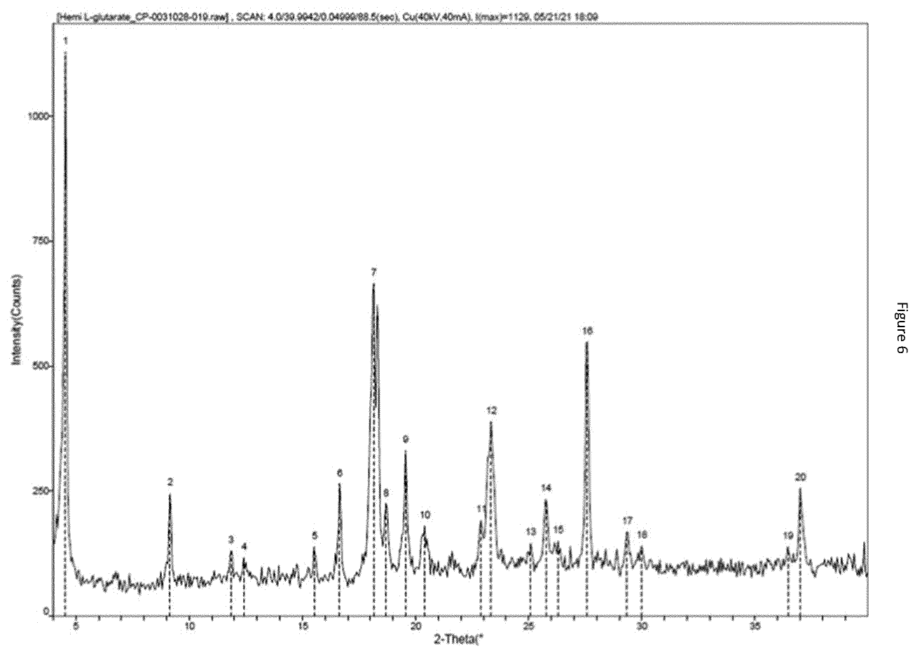

FIG. 6 shows the labelled pattern of the scaled up sample hemi L-glutarate salt of ulodesine, provided and made in accordance with the present disclosure; and

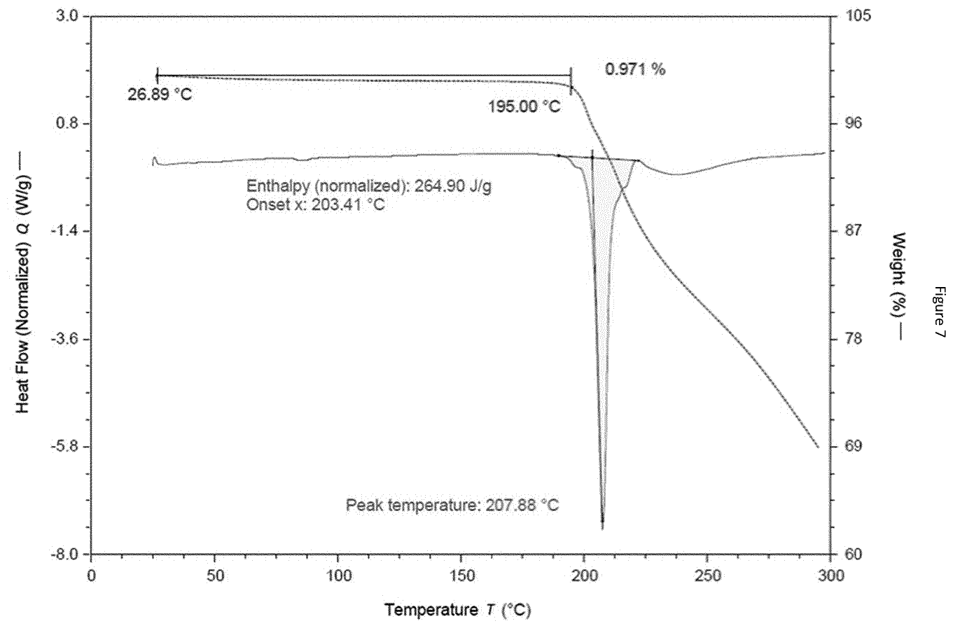

FIG. 7 shows the DSC and TGA overlay of the scaled up sample of hemi L-glutarate salt of ulodesine, provided and made in accordance with the present disclosure.

### DETAILED DESCRIPTION

The precise examples presented herein are for purposes of demonstrating the invention but are not entirely limiting to the compositions or methods of this disclosure.

Identifying and Making Hemi-Salts from Ulodesine (Free)

Structures of the hemi-salts of Ulodesine under potential review are as follows:

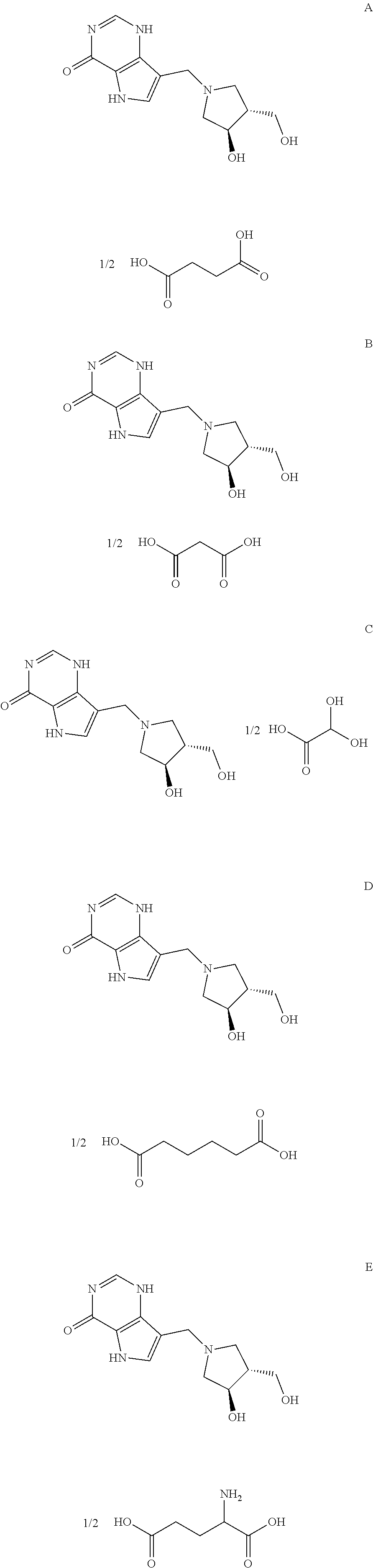

### Methods of Preparation

It was hoped that the freebase Ulodesine, prepared by Pre-HPLC from Ulodesine succinate (A), would be mixed with acids in the ratio of 2:1 in water and then lyophilized to give a preferred hemi-salt form of four potential salts of ulodesine:
Ulodesine Hemi malonate (B),
Ulodesine hemi oxalate (C),
Ulodesine hemi adipinate (D); and
Ulodesine hemi L-glutarate (E).

In each case the resulting salt product was required to be analysed and characterised to elucidate in practice, whether the hemi salt could be reliably produced and in which, if any, desirable structural form.

### Crystalline/Amorphous Salt Form:

The compounds attempting to be made and described in the present disclosure, apart from hemi succinate which is the reference known to be crystal, would potentially exist in a crystalline or non-crystalline (e.g. amorphous) state.

Whether or not a compound exists in a crystalline state can readily be determined by standard techniques and these are defined herein. Crystals and their crystal structures are characterised using a number of techniques including single crystal X-ray crystallography, X-ray powder diffraction (XRPD), differential scanning calorimetry (DSC) and infra-red spectroscopy, e.g. Fourier Transform infra-red spectroscopy (FTIR). The behaviour of the crystals under conditions of varying humidity can be analysed by gravimetric vapour sorption studies and also by XRPD. These techniques help characterise the salts produced and confirm whether the product might be optimised or not appropriate for further investigation.

In particular, X-ray crystallography involves the analysis and interpretation of the X-ray diffraction of single crystal. In an amorphous solid, the three dimensional structure that normally exists in a crystalline form does not exist and the positions of the molecules relative to one another in the amorphous form are essentially random.

To attempt to obtain crystals, the hemi salts for investigation were recrystallized from water and other organic solvents. The disclosure provides solvates formed by the incorporation of a non-toxic pharmaceutically acceptable solvent into the solid-state structure (e.g. crystal structure) of the compounds provided herein. Examples of such solvents may include water, alcohols (such as ethanol, isopropanol and butanol) and dimethylsulfoxide.

Thermogravimetric analysis (TGA), differential scanning calorimetry (DSC) and X-ray crystallography help determine if a solvate has been formed in any given instance. The solvates can be stoichiometric or non-stoichiometric solvates and may include hydrates such as hemihydrates, monohydrates and dihydrates. Alternatively, the resulting compound may be anhydrous (e.g. anhydrous crystalline form).

Only three crystalline hemi salts (Ulodesine Hemi succinate; Ulodesine hemi adipinate; and Ulodesine hemi L-glutarate) were successfully obtained and the hemi L-glutarate was particularly challenging to obtain. Of these, only the reference salt and method of synthesizing was known from the art and it was not possible to achieve the L-glutarate using reference methods.

The yield, NMR and LC-MS analysis of the products was determined for each with parameters as follows: LC-MS Method: Mobile Phase: A: Water (10 mM NH₄HCO3) B: ACN; Gradient: from 5% to 95% of B within 1.3 min.; Flow Rate: 2.0 mL/min; Column: C18 4.6*50 mM, 3.5 μm; Oven Temperature: 40° C. 1H solution NMR was collected on 400 MHz NMR Spectrometer using DMSO-d6 as solvent.

### Ulodesine Hemi Succinate (A)

To a solution of Ulodesine (500.00 mg, 1.89 mmol) in water (30 mL) was added succinic acid (111.70 mg, 0.95 mmol). The mixture was stirred at R.T. for 30 min and then lyophilized to give 556.00 mg of white solid. The yield was 91%. Other batch of hemi succinate (424.00 mg) was prepared from 400.00 mg of Ulodesine. 980.00 mg of Ulodesine hemi succinate salt was dissolved in 3 mL of water, heated to 75° C., and then 30 mL of acetonitrile was added dropwise over 1 h. Then the mixture was cooled to 0° C. over 1 h. The mixture was filtered and the filter cake was washed by acetonitrile and dried to give 702 mg of white solid crystal. The yield of recrystallization was 71.6%.

| Item | Result |
|---|---|
| Yield | 71.6% |
| LC-MS | 100% purity (214 nm), [M + H]⁺ = 265.0, Ret: 0.42 min. |
| ¹H NMR | Consistent and clean; |
| | 1H NMR (400 MHz, DMSO-d6): δ 12.05 (s, 1H), 11.88 (s, 1H), 7.81 (s, 1H), 7.32 |
| | (d, J = 2.4 Hz, 1H), 4.80 (br, 1H), 3.82-3.77 (m, 1H), 3.73-3.65(m, 2H), 3.55- |
| | 3.49(m, 1H), 3.25-3.23(m, 1H), 2.83-2.76(m, 2H), 2.49-2.47 (m, 1H), 2.34 (s, |
| | 2H), 1.97-1.96 (m, 1H). |

### Ulodesine Hemi Adipinate (D)

To a solution of Ulodesine (910.00 mg, 3.44 mmol) in water (50 mL), adipic acid (251.60 mg, 1.72 mmol) was added. The mixture was stirred at R.T. for 30 min and then lyophilized to give 1056.00 mg of white solid. The yield was 91%. 1056 mg of Ulodesine hemi adipinate salt was dissolved in 5 mL of water, heated to 75° C., and then 30 mL of acetonitrile was added dropwise over 1 h. Then the mixture was cooled to 0° C. over 30 min. The mixture was filtered and the filter cake was washed by acetonitrile and dried to give 920 mg of white solid crystal. The yield was 87.1%.

| Item | Result |
|---|---|
| Yield | 87.1% |
| LC-MS | 100% purity (214 nm), [M + H]⁺ = 265.0, Ret: 0.42 min. |
| ¹H NMR | Consistent and clean; |
| | 1H NMR (400 MHz, DMSO-d6): δ 11.96 (s, 1H), 11.83 (s, 1H), 7.78 (s, 1H), 7.26 |
| | (d, J = 2.8 Hz, 1H), 4.80 (br, 1H), 3.74-3.73 (m, 1H), 3.63-3.54(m, 2H), 3.43- |
| | 3.39(m, 1H), 3.25-3.21(m, 1H), 2.68-2.64(m, 2H), 2.37-2.34 (m, 1H), 2.29-2.25 |
| | (m, 1H), 2.19-2.18 (m, 2H), 1.97-1.96 (m, 1H). 1.50-1.48 (m, 2H). |

### Ulodesine Hemi L-Glutarate (E)

### Preliminary Failure:

Ulodesine hemi L-glutarate salt was dissolved in 3 mL of water, heated to 75° C., and then 30 mL of acetonitrile was added dropwise over 1 h. Then the mixture was stirred at this temperature for 1 h. The mixture was cooled to 0° C. over 1 h. The mixture was filtered and the filter cake was washed by acetonitrile and dried to give 320 mg of white solid. 1HNMR showed it was Ulodesine mono L-glutarate salt. The obtained solid and the mother liquid were combined and concentrated, dried by oil pump to give 1055 mg of white solid.

However, the process(with water and acetonitrile etc.) previously used to produce other salts above produced a white solid mono form. The desirable hemi crystal form of the glutarate was not able to be obtained by this method.

New methodology “J” required:

Ultimately, for successful production of Ulodesine hemi L-glutarate in the useful crystal form, several technical variations of the crystallisation process were needed to be investigated.

### Finally, Using a Mixed Solvent Process, and Using Several Different Solvents, a New Crystallisation Method Specific to the Hemi-Glutarate Form (“J”) was Determined:

To a solution of Ulodesine (1000.00 mg, 3.78 mmol) in water (50 mL) L-glutaric acid (278.36 mg, 1.89 mmol) was added. The mixture was stirred at R.T. for 30 min and then lyophilized to give 1155.00 mg of white solid. The yield was 90%.

1055 mg of Ulodesine hemi L-glutarate salt was dissolved in 3 mL of water and then heated to 75° C., 15 mL of ethanol was added and stirred at this temperature for 30 min to form a homogeneous solution. Then 30 mL of acetonitrile was added dropwise over 1 h. Then the mixture was stirred at this temperature for 1 h. The mixture was cooled to 0° C. over 1 h. The mixture was filtered and the filter cake was washed by acetonitrile and dried to give 810 mg of Ulodesine hemi L-glutarate as white solid.

The yield was 76.8%.

Analysis (see further below) determined this solid was crystal hemi L-glutarate and confirmed that a new alternative method, including several steps and the addition of ethanol, was necessary for effective recrystallization of Ulodesine hemi L-glutarate.

In a particular embodiment, the present disclosure therefore concerns Ulodesine hemi L-glutarate, formed using the process of re-crystallisation as described herein.

| Item | Result |
|---|---|
| Yield | 76.8% |
| LC-MS | 100% purity (214 nm), [M + H]⁺ = 265.0, Ret: 0.42 min. |
| ¹H NMR | Consistent and clean; |
| | 1H NMR (400 MHz, DMSO-d6): δ 11.98 (s, 1H), 11.83 (s, 1H), 7.79 (s, 1H), 7.27 (s, |
| | 1H), 4.80 (br, 1H), 3.78-3.74 (m, 1H), 3.67-3.58(m, 2H), 3.43-3.40(m, 2H), 3.33- |
| | 3.21(m, 2H), 2.72-2.68(m, 2H), 2.49-2.30 (m, 3H), 1.97-1.96 (m, 1H), 1.92-1.88 (m, |
| | 1H). |

### Amorphous Salts:

The other two hemi salts (Ulodesine hemi malonate and Ulodesine hemi oxalate) were amorphous. Water and acetonitrile, tetrahydrofuran, ethanol were used for the recrystallization of these two hemi salts, but none of the obtained solid was crystal.

### Ulodesine Hemi Malonate (B)

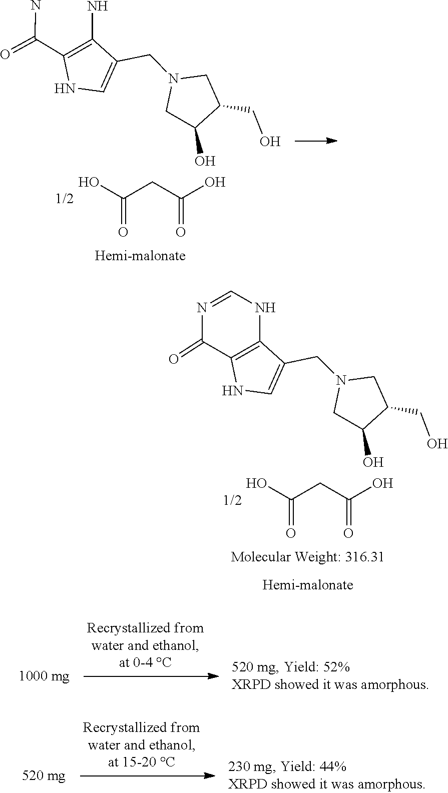

To a solution of Ulodesine (1000.00 mg, 3.78 mmol) in water (50 mL) malonic acid (196.87 mg, 1.89 mmol) was added. The mixture was stirred at R.T. for 30 min and then lyophilized to give 1103 mg of white solid.

1103 mg of Ulodesine hemi malonate salt was dissolved in 10 mL of water and then heated to 75° C., then 40 mL of acetonitrile was added dropwise over 1 h. Then the mixture was stirred at this temperature for 1 h. The mixture was cooled to 0° C. over 1 h. The mixture was filtered and the filter cake was washed by acetonitrile and dried to give 625 mg of white solid. The yield was 56.7%. XRPD showed it was amorphous. The filtrate was concentrated in vacuum and dried by oil pump. The obtained solid and the amorphous form were combined to give 1025 mg of white solid.

1000 mg of Ulodesine hemi malonate salt was dissolved in 5 mL of water, heated to 75° C., and then 16 mL of ethanol was added and stirred at this temperature for 30 min to form a homogeneous solution. Then 40 mL of acetonitrile was added dropwise over 1 h. Then the mixture was stirred at this temperature for 1 h. The mixture was cooled to 0° C., over 1 h. The mixture was filtered and the filter cake was washed by acetonitrile and dried to give 715 mg of white solid. The yield was 71.5%. XRPD showed it was amorphous. The filtrate was concentrated in vacuum and dried by oil pump. The obtained solid and the amorphous form were combined to give 980 mg of white solid.

1000 mg of Ulodesine hemi malonate salt was dissolved in 5 mL of water, heated to 75° C., and then 25 mL of tetrahydrofuran was added dropwise over 1 h. Then the mixture was stirred at this temperature for 1 h. The mixture was cooled to 0° C., over 1 h. No solid appeared. No solid was obtained after standing at 0-4° C. for 3 days. The mixture was concentrated in vacuum and dried by oil pump to give 1000 mg of white solid.

1000 mg of Ulodesine hemi malonate salt was dissolved in 5 mL of water and then heated to 75° C., then 25 mL of ethanol was added dropwise over 1 h. Then the mixture was stirred at this temperature for 1 h. The mixture was cooled to 0° C., over 1 h. The mixture was stood at room temperature for 1 day. The mixture was filtered and the filter cake was washed by ethanol and dried to give 520 mg of white solid. The yield was 52.0%. XRPD showed it was amorphous. The filtrate was concentrated in vacuum and dried by oil pump. The obtained solid and the amorphous form were combined to give 969 mg of white solid

| Item | Result |
|---|---|
| Yield | 52.0% |
| LC-MS | 100% purity (214 nm), [M + H]⁺ = 265.0, Ret: 0.41 min. |
| ¹H NMR | Consistent and clean; |
| | 1H NMR (400 MHz, DMSO-d6): δ 12.17 (s, 1H), 11.93 (s, 1H), 7. 84(s, 1H), 7.39 |
| | (s, 1H), 3.90 (s, 3H), 3.45-3.40 (m, 1H), 3.28(br, 1H), 3.00 (br, 2H), 2.76 (s, 1H), |
| | 2.68 (br, 2H), 1.91 (s, 1H). |

### Ulodesine Hemi Oxalate (C)

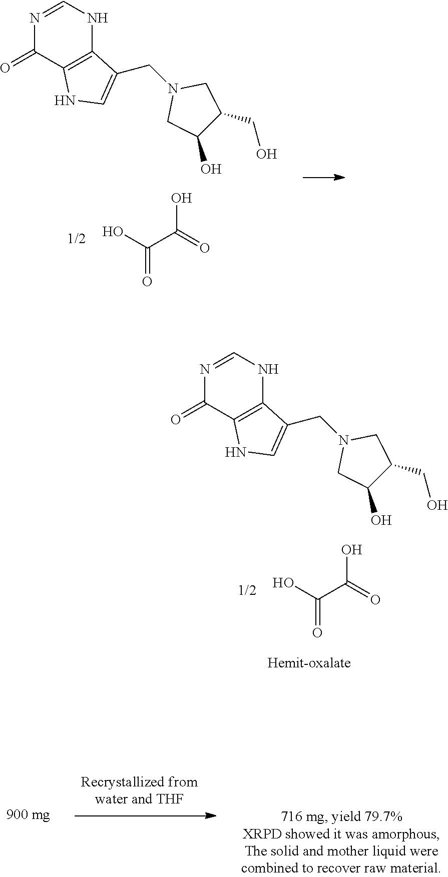

To a solution of Ulodesine (1000.00 mg, 3.78 mmol) in water (50 mL), oxalic acid (170.33 mg, 1.89 mmol) was added. The mixture was stirred at R.T. for 30 min and then lyophilized to give 1045 mg of white solid.

1045 mg of Ulodesine hemi oxalate salt was dissolved in 10 mL of water, heated to 75° C., and then 40 mL of acetonitrile was added dropwise over 1 h. Then the mixture was stirred at this temperature for 1 h. The mixture was cooled to 0° C. over 1 h. The mixture was filtered and the filter cake was washed by acetonitrile and dried to give 712 mg of white solid. The yield was 68.0%. XRPD showed it was amorphous. The filtrate was concentrated in vacuum and dried by oil pump. The obtained solid and the amorphous were combined to give 915 mg of white solid.

900 mg of Ulodesine hemi oxalate salt was dissolved in 5 mL of water, heated to 75° C., and then 16 mL of ethanol was added and stirred at this temperature for 30 min to form a homogeneous solution. Then 40 mL of acetonitrile was added dropwise over 1 h. Then the mixture was stirred at this temperature for 1 h. The mixture was cooled to 0° C. over 1 h. The mixture was filtered and the filter cake was washed by acetonitrile and dried to give 705 mg of white solid. The yield was 77.7%. XRPD showed it was amorphous. The filtrate was concentrated in vacuum and dried by oil pump. The obtained solid and the amorphous were combined to give 900 mg of white solid.

900 mg of Ulodesine hemi oxalate salt was dissolved in 5 mL of water, heated to 75° C., and then 25 mL of tetrahydrofuran was added dropwise over 1 h. Then the mixture was stirred at this temperature for 1 h. The mixture was cooled to 0° C. over 1 h. The mixture was filtered and the filter cake was washed by acetonitrile and dried to give 716 mg of white solid. The yield was 79.7%. XRPD showed it was amorphous. The filtrate was concentrated in vacuum and dried by oil pump. The obtained solid and the amorphous form were combined to give 880 mg of white solid.

| Item | Result |
|---|---|
| Yield | 68.0% |
| LC-MS | 100% purity (214 nm), [M + H]⁺ = 265.0, Ret: 0.42 min. |
| ¹H NMR | Consistent and clean; |
| | 1H NMR (400 MHz, DMSO-d6): δ 12.23 (s, 1H), 11.99 (s, 1H), 7.86 (s, 1H), 7.43 |
| | (d, J = 2.8 Hz, 1H), 3.99 (d, J = 20.4 Hz, 3H), 3.45-3.40 (m, 1H), 3.39-3.27(m, 1H), |
| | 3.11 (d, J = 6.4 Hz, 2H), 2.78 (d, J = 2.8 Hz, 2H), 2.08 (s, 1H). |

### Characterisation Study of Salt Forms

New salts, made for the first time, in particular, the hemi glutarate, hemi adipinate and two other newly formed salts (which were amorphous) were compared in character/property to a known salt. These previously uncharacterised salts of ulodesine (adipinate, glutarate, malonate and oxalate) were ultimately prepared in accordance with the methods above. An exemplary pharmaceutically acceptable salt of ulodesine, hemi succinate, was selected for comparison and made in accordance with known methods in the art.

The characteristics and properties were determined herein using standard analytical means according to the following section of explanation:

### Analytical Techniques

The discussion herein is aided by the drawing in graphic form of various analysis techniques including Polarized light microscope (PLM), the X-ray powder diffraction (XRPD), differential scanning calorimetry (DSC), and thermogravimetric analysis (TGA).

### Polarized Light Microscope (PLM)

Place small amount of sample (<1 mg) on glass slide, add a drop of liquid paraffin, and cover with slip. The sample dispersed in oil is observed through microscope eyepiece and camera/computer system.

A representative sample image will be captured and annotated to measure particle size and crystal habit.

### X-Ray Powder Diffraction (XRPD)

XRPD is a technique used on powder or microcrystalline samples for structural characterization of materials.

X-ray powder diffraction (XRPD) patterns were obtained on a Bruker D8 Advance with a CuK source (1.54056 angstrom) operating minimally at 40 kV and 40 mA scans for each sample between 4 and 40 degrees 2-theta. The 2-theta step size is 0.05 and scan speed is 0.5 s/step.

### Differential Scanning Calorimetry (DSC)

DSC is a thermoanalytical technique in which the difference in the amount of heat required to increase the temperature of a sample and reference are measured as a function of temperature.

Differential scanning calorimetry analyses were carried out on a TA Instrument DSC unit (Model DSC 25). Samples were heated in non-hermetic aluminium pans from ambient to 300° C. at 10° C./min with a nitrogen purge of 50 mL/min.

### Thermogravimetric Analyses (TGA)

TGA is a type of testing that is performed on samples to determine changes in weight in relation to changes in temperature.

Thermogravimetric analyses were carried out on a TA Instrument TGA unit (Model: TGA 500). Samples were heated in platinum pans from ambient to 300° C. at 10° C./min with a nitrogen purge of 60 mL/min (sample purge) and 40 mL/min (balance purge).

### Dynamic Vapour Sorption (DVS)

The moisture sorption profile was generated at 25° C. using a DVS moisture balance flow system (Model Advantage 1.0) with the following conditions: sample size approximately mg, drying 25° C. for 60 minutes, adsorption range 0% to 95% RH, desorption range 95% to 0% RH, and step interval 5%. The equilibrium criterion was <0.01% weight change in 5 minutes for a maximum of 120 minutes.

### Water Content (KF)

Three blank samples were measured in parallel, and then ~10 mg samples (two samples were tested in parallel) were weighed for testing by Karl-Fischer titration-Volumetric method.

### 1H NMR

1H solution NMR was collected on Bruker 400 MHz NMR Spectrometer using DMSO-d6 as solvent.

The properties of the crystal and amorphous forms are summarised below in the discussion and tables that follow:

### Limited Characterisation of Amorphous Salts

Summary table

| | Sample name | | | |
|---|---|---|---|---|
| | Hemi-oxalate | | Hemi L-malonate | |
| Lot | CP-0031028-015-OX | CP-0031028-016-OX | CP-0031028-015-LG | CP-0031028-016-MA |
| Crystallization | ACN and water | ACN, ethanol and water | ACN and water | ACN, ethanol and water |
| solvent | | | | |
| Appearance | White powder | White powder | White powder | White powder |
| XRPD | Very Poor crystallinity | Amorphous | Amorphous | Amorphous |

### Ulodesine Hemi Malonate

The two batches of Ulodesine hemi malonate XPRD pattern indicated that the compound was amorphous with no distinct diffraction peaks, so no further characterization was performed.

### Ulodesine Hemi Oxalate

The XPRD pattern indicated that one batch was poor crystalline, close to amorphous with only one small distinct diffraction peak, the other batch was amorphous so no further characterisation was undertaken.

### Characterisation of Crystal Salts

Following clear indication of a crystal form for hemi succinate, hemi adipinate and hemi L-glutarate, further characterisation studies were carried out and solubility was determined thereafter.

The following summary table displays the full results of the further analytical studies undertaken to characterise the three crystal salts:

| | Ulodesine hemi | Ulodesine hemi | Ulodesine hemi |
|---|---|---|---|
| Sample Name | adipinate | succinate | L-glutarate |
| Appearance | White powder | White solid | White solid |
| XRPD | Crystal | Crystal | Crystal |
| PLM | Birefringence with | Birefringence with | Birefringence with |
| | particles and rods | particles and rods | particles and |
| | morphology | morphology | irregular blocks |
| | | | morphology |
| DSC | Endo. = 70.46° C.; | Endo. = 75.95° C.; | Melting point = |
| | Endo. = 97.50° C.; | Endo. = 178.34° C.; | 190.03° C. |
| | Melting point = 128.21° C. | Exo. = 183.64° C. | |
| | | Melting point = | |
| | | 204.48° C. | |
| TGA | 5.041% weight loss | 5.593% weight loss | 2.637% weight loss |
| | (from RT to 128° C.) | (from RT to 120° C.) | (from RT to 190° C.) |
| Molar ratio | 2:1(by NMR) | 2:1(by NMR) | 2:1(by NMR) |
| (Free base:acid) | | | |
| Purity | 100% (by LCMS) | 100% (by LCMS) | 100% (by LCMS) |
| Water content | 5.34% | 6.03% | 2.53% |
| DVS | Moderately hygroscopic | Moderately hygroscopic | Moderately hygroscopic |
| | (3.68% at 80% RH; | (6.97% at 80% RH; | (2.53% at 80% RH; |
| | 15.80% at 95% RH) | 11.86% at 95% RH) | 27.06% at 95% RH) |
| XRPD pre and | No change | No change | No change |
| post DVS | | | |
| Speculated form | Monohydrate | Monohydrate | Anhydrate |
| Solubility | Water: 198.2 mg/mL | Water: 120.4 mg/mL | Water: 93.4 mg/mL |
| (37° C., 24 hr) | SGF(pH 1.2): 170.6 | SGF(pH 1.2): 106.2 | SGF(pH 1.2): 131.5 |
| | mg/mL | mg/mL | mg/mL |
| | FaSSIF (pH 6.5): | FaSSIF (pH 6.5): 114.7 | FaSSIF (pH 6.5): |
| | 120.4 mg/mL | mg/mL | 101.8 mg/mL |
| | FeSSIF (pH 5): | FeSSIF (pH 5): 117.1 | FeSSIF (pH 5): 111.0 |
| | 125.7 mg/mL | mg/mL | mg/mL |
| Solid stability | N/A | 0.34% TRS increased at | 0.42% TRS |
| (60° C. (closed); | | 2 weeks under 60° C.; | increased at 2 |
| 40° C./75% RH | | 0.16% TRS increased at | weeks under 60° C.; |
| (open); | | 2 weeks under | No TRS increased at |
| during 2 weeks) | | 40° C./75% RH. | 2 weeks under |
| | | | 40° C./75% RH. |

Ulodesine Hemi-L-glutarate was white solid by visual observation and birefringence and non-birefringence with particles and irregular blocks morphology under PLM. The XPRD pattern indicated that the compound was crystal as shown in FIG. 1 but possibly with poor crystallinity, which was consistent with the PLM result. The information showing the distinct diffraction peaks is provided below:

| | |
|---|---|
| [Hemi L-glutarate CP-0031028-016-LG.raw] | Peak Search Report |

SCAN: 4.0/39.9942/0.04999/88.5(sec), Cu(40 kV, 40 mA), t(max) = 308, Mar. 16, 2021 17:25
PEAK: 13-pts/Parabolic Filter, Threshold = 3.0, Cutoff = 0.1%, BG = 3/1.0, Peak-Top = Summit
NOTE: Intensity = Counts, 2T(0) = 0.0(°), Wavelength to Compute d-Spacing = 1.54056 A(Cu/K-alpha1)

| # | 2-Theta | d(A) | BG | Height | I % | Area | I % | FWHM |
|---|---|---|---|---|---|---|---|---|
| 1 | 4.542 | 19.4406 | 92 | 187 | 88.6 | 971 | 42.1 | 0.221 |
| 2 | 11.890 | 7.4370 | 74 | 35 | 16.6 | 60 | 2.6 | 0.069 |
| 3 | 15.572 | 5.6858 | 76 | 49 | 23.2 | 154 | 6.7 | 0.134 |
| 4 | 16.645 | 5.3217 | 81 | 74 | 35.1 | 246 | 10.7 | 0.141 |
| 5 | 18.066 | 4.9090 | 91 | 194 | 91.9 | 2308 | 100.0 | 0.506 |
| 6 | 18.701 | 4.7408 | 106 | 87 | 41.2 | 667 | 28.9 | 0.326 |
| 7 | 19.556 | 4.5352 | 103 | 156 | 73.9 | 681 | 29.5 | 0.185 |
| 8 | 20.325 | 4.3656 | 100 | 47 | 22.3 | 184 | 8.0 | 0.166 |
| 9 | 23.366 | 3.8058 | 97 | 211 | 100.0 | 1882 | 81.5 | 0.379 |
| 10 | 25.839 | 3.4452 | 109 | 41 | 19.4 | 264 | 11.4 | 0.274 |
| 11 | 27.548 | 3.2351 | 107 | 52 | 24.6 | 322 | 14.0 | 0.263 |
| 12 | 29.485 | 3.0269 | 100 | 38 | 18.0 | 202 | 8.8 | 0.226 |

As shown in FIG. 2, the DSC trace displayed one single melting point at 190.03° C. 2.637% weight loss was found from RT to 190° C. under TGA curve that may due to desolvate of water because the water content determined by Karl-Fischer titration was 2.53%. The Ulodesine hemi-L-glutarate was moderately hygroscopic, 2.53% of water uptake was observed from 0% RH to 80% RH at 25° C. on DVS plot and the crystal form displayed no change, pre- and post-DVS as shown in the XRPD overlay of FIG. 3. Based on the result of DSC, TGA and water content, Ulodesine hemi-L-glutarate was speculated to be anhydrate.

### Solubility Study

The thermodynamic solubility study was conducted on the three crystal Ulodesine salts in water, SGF, FaSSIF and FeSSIF under RT (21~25° C.) for 24 hrs. The solubility study procedure was as follow: appropriate amount of each salt were weighed into 2 mLglass vial, respectively, followed by addition of 0.4 mL desired vehicles (water, SGF, FaSSIF and FeSSIF) to form suspensions. Then all the samples were vortex for 30 s to disperse homogenously and placed into a constant temperature shaking incubator at 37° C. with 200 rpm for 24 hrs. If the API dissolved, more material will be added to keep suspension state. The concentration, XRPD and pH were tested at desired time points. A summary of the results is provided below:

| | | | pH value | | |
|---|---|---|---|---|---|
| Sample | Vehicle | Solubility(mg/mL) | at 24 hrs | XRPD | Appearance |
| Hemi | water | 198.2 | 6.56 | Not | A few |
| adipinate | | | | enough | particles |
| | SGF(pH 1.2) | 170.6 | 5.60 | residual | A few |
| | | | | solid | particles |
| | FaSSIF(pH 6.5) | 120.4 | 6.40 | | A few |
| | | | | | particles |
| | FeSSIF(pH 5) | 125.7 | 6.09 | | A few |
| | | | | | particles |
| Hemi | water | 120.4 | 6.34 | | A few |
| succinate | | | | | particles |
| | SGF(pH 1.2) | 106.2 | 5.32 | | A few |
| | | | | | particles |
| | FaSSIF(pH 6.5) | 114.7 | 6.16 | | A few |
| | | | | | particles |
| | FeSSIF(pH 5) | 117.1 | 5.75 | | A few |
| | | | | | particles |
| Hemi L- | water | 93.4 | 7.58 | Change | Turbid, |
| glutarate | | | | to | many |
| | | | | freebase | particles |
| | SGF(pH 1.2) | 131.5 | 7.75 | Not | Turbid, a |
| | | | | enough | few |
| | | | | residual | particles |
| | | | | solid | |
| | FaSSIF(pH 6.5) | 101.8 | 7.72 | Change | Turbid, |
| | | | | to | many |
| | | | | freebase | particles |
| | FeSSIF(pH 5) | 111.0 | 7.78 | Change | Turbid, |
| | | | | to | many |
| | | | | freebase | particles |

Ulodesine hemi adipinate, Ulodesine hemi succinate, and Ulodesine hemi L-glutarate all showed high solubility (~100 mg/mL) under 37° C. at 24 hrs. FIG. 4 shows the HPLC overlay of Ulodesine Hemi-L-glutarate samples in solubility study in the four vehicles (water, SGF, FaSSIF, FeSSIF).

The XRPD pattern of residual solid of Ulodesine hemi L-glutarate in water, FaSSIF and FeSSIF was changed compared to initial solid form, and it was proved to be freebase (Ulodesine) based on the 1H NMR result of residual solid in FaSSIF after solubility.

### Solid State Stability Study—Hemi L-Glutarate

A further study focussing on the potential for solid state stability was performed to investigate the physical and chemical stability for Ulodesine hemi L-glutarate under 60° C. (closed) and 40° C./75% RH (open) conditions during 2 weeks. This was also undertaken for Ulodesine hemi succinate to provide means for comparison.

The procedure for solid state stability was as follows: 10 mg of Ulodesine hemi succinate and Ulodesine hemi L-glutarate were weighed accurately into a 40 mL clear glass vial, respectively, and then the sample vials were placed into the corresponding condition For the samples opened in humidity condition, open without cap and covered by aluminium foil with pinhole; for the closed samples, they were all capped.

At 0, 1 week and 2 weeks' time points, the corresponding samples will be sampled for purity determination by HPLC to assess the chemical stability. Also, one more sample was prepared under corresponding condition for appearance and XRPD test to evaluate the physical stability.

The result of solid stability study showed that no appearance and XRPD changed for the two salts during 2 weeks under 60° C. and 40° C./75% RH conditions. The HPLC study results for hemi L-glutarate are presented in FIG. 5. For 60° C. condition, slightly degradation (0.3%-0.4%) was observed on both salts at 2 weeks' time point. For 40° C./75% RH condition, no related substance increased on hemi L-glutarate, while hemi succinate was degraded 0.16-0.3% during 2 weeks.

Based on the purity result, it seems Ulodesine hemi L-glutarate was more chemically stable than Ulodesine Hemi succinate under 40° C./75% RH condition.

The results are shown in table summary below:
*: XRPD: Compared to the XRPD pattern at initial time.

| | | | | | | TRS(%) | TRS Change(%) |
|---|---|---|---|---|---|---|---|
| | | Time | | | | (=100% − | (Compared to |
| Sample | Condition | point | Appearance | XRPD* | Purity(%) | purity %) | 0 d) |
| Ulodesine | // | 0 d | White | // | 99.942 | 0.06 | // |
| Hemi | | | solids | | | | |
| succinate | 60° C. | 1 week | White | No | 99.816 | 0.18 | 0.13 |
| | close | | solids | change | | | |
| | | 2 weeks | White | No | 99.601 | 0.40 | 0.34 |
| | | | solids | change | | | |
| | 40° C./ | 1 week | White | No | 99.613 | 0.39 | 0.33 |
| | 75% RH | | solids | change | | | |
| | open | 2 weeks | Pale yellow | No | 99.782 | 0.22 | 0.16 |
| | | | block solids | change | | | |
| Ulodesine | // | 0 d | White | // | 99.403 | 0.60 | // |
| Hemi L- | | | solids | | | | |
| glutarate | 60° C. | 1 week | White | No | 99.258 | 0.74 | 0.15 |
| | close | | solids | change | | | |
| | | 2 weeks | White | No | 98.984 | 1.02 | 0.42 |
| | | | solids | change | | | |
| | 40° C./ | 1 week | White | No | 99.524 | 0.48 | −0.12 |
| | 75% RH | | solids | change | | | |
| | open | 2 weeks | White | No | 99.600 | 0.40 | −0.20 |
| | | | solids | change | | | |

The hemi L-glutarate has good chemical and physical stability comparing very favourably with the hemi succinate having with no appearance and polymorph change for 2 weeks under 60° C. and 40° C./75% RH conditions. The two salts had comparable and very slight degradation (0.3%-0.4%) in the case of 60° C. condition for 2 weeks.

In summary, the applicant has determined that Ulodesine hemi L-glutarate salt was more chemically stable than Ulodesine hemi succinate overall.

The applicant therefore concluded that despite initial challenges, hemi-glutarate crystal form is able to be successfully made and demonstrates good physical and chemical properties relative to available salts of ulodesine.

It could therefore be a useful candidate in pharmaceutical formulation processing.

Further stability was investigated e.g. within likely example formulation types e.g. aqueous and potential for good consistent yield for scaling up production for batch testing.

### Hemi L-Glutarate Salt—Aqueous Formulation Stability

A further study focussed on the newly-characterised salt was conducted to provide stability data for supporting aqueous intravenous (IV) formulations.

The formulation comprised Ulodesine hemi L-glutarate salt in pure water at a solution concentration of 0.1 mg/ml. The sample condition was maintained at 21-25° C. and protected from light.

The study procedure was as follows: 6.4 mg of the compound Ulodesine hemi L-glutarate was weighed into a 50 mL volumetric flask, sonicated until dissolved and diluted to the volume, the target concentration was 0.10 mg/ml (cal. by freebase).

Duplicate samples were prepared by transfer 4 ml solution into an 8 mL glass bottle and all the samples were placed in dark under room temperature. At desired time points of 0, 3, 7, 10 and 14 days, the sample concentration was analysed by HPLC and the pH value was measured.

The result showed no significant change was observed on appearance, concentration and purity of Ulodesine hemi L-glutarate solution samples in water at 0.1 mg/ml (cal. by freebase) during 14 days at room temperature. The formulation was physically and chemically stable for 14 days.

In summary, it was determined that ulodesine hemi L-glutarate showed good formulation stability in water at 0.1 mg/mL (calculated by freebase) under room temperature for 14 days and could likely be scaled up, which may be useful to support animal studies, especially within Intra-Venous (IV) formulations.

### Repetition of Salt-Forming Process (for 1 g Scale-Up)

Firstly, the process of crystal salt production of the hemi-glutarate form was repeated to determine if the provision of the salt, from the free form, using this newly established method was reliable and consistent to yield enough of the correctly identified and pure product to be useful to scale up. The initial aim was to obtain 1 g of the desired product and determine consistency with the earlier analytical product data.

To a solution of 1g Ulodesine (1000.00 mg, 3.78 mmol) in water (50 mL), L-glutaric acid (278.36 mg, 1.89 mmol) was added. The mixture was stirred at R.T. for 30 min and then lyophilized to give 1170.00 mg of white solid. 1170 mg of Ulodesine hemi L-glutarate salt was dissolved in 5 mL of water and then heated to 75° C., 16 mL of ethanol was added and stirred at this temperature for 30 min to form a homogeneous solution. Then 40 mL of acetonitrile was added dropwise over 1 h. Then the mixture was stirred at this temperature for 1 h. The mixture was cooled to 0° C., over 1 h. The mixture was filtered and the filter cake was washed by acetonitrile and dried to give 1010 mg (1 g) of white solid. The yield was 79.0%. The final material is first confirmed by LC-MS and NMR as below:

| Item | Result |
|---|---|
| Yield | 79.0% |
| LC-MS | 100% purity (214 nm), [M + H]⁺ = 265.0, Ret: 0.77 min. |
| ¹H NMR | Consistent and clean; |
| | 1H NMR (400 MHz, D2O): 7.87 (s, 1H), 7.48 (s, 1H), δ 4.08 (dt, J = 5.9, 3.7 Hz, |
| | 1H), δ 4.05 (s, 2H), 3.59 (dd, J = 7.2, 4.7 Hz, 0.5H), 3.44 (qd, J = 11.3, 6.4 Hz, |
| | 2H), 3.27 (dd, J = 11.2, 8.3 Hz, 1H), 3.08 (dd, J = 11.7, 5.9 Hz, 1H), 2.93 (dd, J = |
| | 11.7, 3.4 Hz, 1H), 2.69 (dd, J = 11.3, 7.2 Hz, 1H), 2.24-2.13 (m, 2H), 2.05-1.88 |
| | (m, 1H). |

It was then characterised as before by PLM, XRPD, DSC and TGA.

Ulodesine hemi L-glutarate was white solids by visual observation and birefringence with particles and irregular blocks morphology under PLM.

The XPRD pattern shown in FIG. 6 indicated that the final compound was crystal with distinct diffraction peaks and the polymorph of this scaled up (1 g) hemi L-glutarate was same as the hemi L-glutarate obtained in preliminary studies referred to in the tables above. Information on the distinct diffraction peaks is found in the table below:

| | |
|---|---|
| [20210521064(Hemi L-glutarate).raw] | Peak Search Report |

SCAN: 4.0/39.9942/0.04999/88.5(sec), Cu(40 kV, 40 mA), t(max) = 1129, May 21, 2021 18:09
PEAK: 11-pts/Parabolic Filter, Threshold = 3.0, Cutoff = 0.1%, BG = 3/1.0, Peak-Top = Summit
NOTE: Intensity = Counts, 2T(0) = 0.0(°), Wavelength to Compute d-Spacing = 1.54056 A(Cu/K-alpha1)

| # | 2-Theta | d(A) | BG | Height | I % | Area | I % | FWHM |
|---|---|---|---|---|---|---|---|---|
| 1 | 4.541 | 19.4428 | 73 | 1056 | 100.0 | 4455 | 93.7 | 0.179 |
| 2 | 9.147 | 9.6600 | 66 | 178 | 16.9 | 610 | 12.8 | 0.146 |
| 3 | 11.846 | 7.4548 | 80 | 49 | 4.6 | 154 | 3.2 | 0.134 |
| 4 | 12.424 | 7.1185 | 74 | 41 | 3.9 | 187 | 3.9 | 0.194 |
| 5 | 15.532 | 5.7003 | 77 | 59 | 5.6 | 143 | 3.0 | 0.103 |
| 6 | 16.643 | 5.3222 | 72 | 191 | 18.1 | 645 | 13.6 | 0.143 |
| 7 | 18.142 | 4.8857 | 87 | 578 | 54.7 | 4757 | 100.0 | 0.350 |
| 8 | 18.697 | 4.7420 | 106 | 117 | 11.1 | 573 | 12.0 | 0.208 |
| 9 | 19.551 | 4.5368 | 99 | 231 | 21.9 | 856 | 18.0 | 0.157 |
| 10 | 20.391 | 4.3517 | 97 | 81 | 7.7 | 342 | 7.2 | 0.179 |
| 11 | 22.898 | 3.8806 | 93 | 97 | 9.2 | 869 | 18.3 | 0.381 |
| 12 | 23.344 | 3.8075 | 96 | 292 | 27.7 | 2540 | 53.4 | 0.370 |
| 13 | 25.084 | 3.5472 | 104 | 40 | 3.8 | 143 | 3.0 | 0.152 |
| 14 | 25.760 | 3.4556 | 106 | 126 | 11.9 | 792 | 16.6 | 0.267 |
| 15 | 26.297 | 3.3862 | 102 | 47 | 4.5 | 392 | 8.2 | 0.354 |
| 16 | 27.587 | 3.2307 | 128 | 420 | 39.8 | 1435 | 30.2 | 0.145 |
| 17 | 29.349 | 3.0406 | 102 | 65 | 6.2 | 315 | 6.6 | 0.205 |
| 18 | 29.995 | 2.9767 | 97 | 41 | 3.9 | 295 | 6.2 | 0.306 |
| 19 | 36.454 | 2.4626 | 99 | 38 | 3.6 | 266 | 5.6 | 0.297 |
| 20 | 37.010 | 2.4269 | 96 | 159 | 15.1 | 846 | 17.8 | 0.225 |

As seen in FIG. 7, the DSC trace displayed one single melting point at 203.41° C. Only 0.971% of weight loss was observed from room temperature to 195° C. on TGA curve. The 1 g hemi L-glutarate product was determined likely be an anhydrate. Further, the crystallinity was a marked improvement from the initial crystallisation, further evidencing that this salt makes for a useful candidate in further formulation testing. Following the successful characterisation and selection of the new salt (Hemi-glutarate from the free form of Ulodesine) and a validation that the recrystalisation method used resulted in this product with the same consistent physical and chemical solid stability, the complete method of manufacture was investigated.

### Complete Method of Manufacture of Ulodesine Hemi-Glutarate

It was necessary to follow a complete ulodesine production process including the re-crystallisation of the free-form product to produce the desired salt in order to confirm that a complete method of manufacture disclosed resulted in the same useful salt product (rather than starting from the simple free base of ulodesine).

In particular, it was important that the final hemi-glutarate salt are reliably obtained by such a method with a view that the final method could be used to obtain larger demonstration batches (suitable for pharmacological processing).

Of particular note, given the previous technical challenges reported in the art, it would be important to establish that the free form of ulodesine could be reliably made as it is essential to efficient and reliable recrystalisation of the newly disclosed hemi-glutarate.

Firstly, the identification of complete process was established. Below is a scheme showing the overall processing steps for the complete synthetic production of the Ulodesine hemi-glutarate salt CG689J of the invention from basic components:

### Synthetic Route:

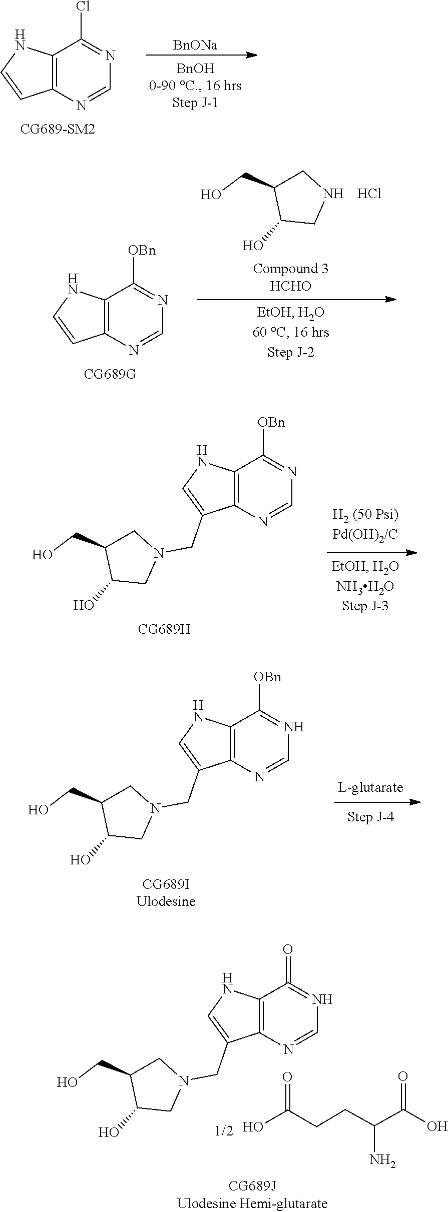

The applicant initially followed the steps above (and described hereinbefore) with reference to the above scheme conditions:

### Step J-1

Hydroxybenzylation was carried out in accordance with known methods.

A polymer of the CG689-SM2 (see above) was observed in all results and the primary reason for yield loss. The applicant made preliminary to optimise the base and replace BnOH as the solvent, as polymer formation took place during the prolonged solvent removal process, however ultimately, a better solvent could not be identified.

The polymer impurity was removed via hot filtration of CG689G and the desired intermediate was obtained in ~40% yield.

### Step J-2

The HCl salt of Compound 3 was used and Mannich reaction was carried out in the presence of K2CO-3.

### Step J-3

The benzyl-protecting group on CG689H was successfully removed after 72 hours under hydrogenolysis conditions. However, the challenge remained that despite the addition of basic additives (ammonium hydroxide solution), Ulodesine was still obtained as a hydrochloride salt rather than the preferred free base, for which it was desirable to produce the new salt directly.

After treatment with ion-exchange resin, Ulodesine free form was obtained in ~33% yield (across two steps) with high HPLC purity and chiral purity (98.3%, 99.7% respectively)

### Step J-4

The final step was undertaken in accordance with the earlier described re-crystallisation process of the hemi-glutarate salt formation. However, although the Ulodesine hemi-glutarate was typically obtained the incomplete dissociation of the hydrochloride salt led to a mixture of salt forms. Upon examination with DSC calorimeter and XRPD, the results do not match up with the findings from hemi-glutarate produced in the salt-selection analysis study described earlier. It was concluded that alternative steps within the manufacture, prior to recrystalisation, would be essential in order to yield the correct salt form with comparable characterisation.

A further technical review of the Ulodesine production method, as per the scheme above, was undertaken. The applicant noted Ulodesine hydrochloride compound 3 is a key component in the earlier production of Ulodesine CG6891 free form. Successfully yielding the free base of Ulodesine was identified as an important factor.

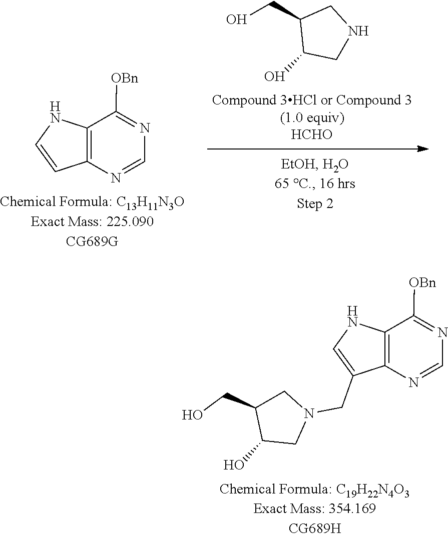 [SMILES: OC[C@H]1CNC[C@@H]1O ; c1ccc(COc2ncnc3cc[nH]c23)cc1 ; OC[C@H]1CN(Cc2c[nH]c3c(OCc4ccccc4)ncnc23)C[C@@H]1O]

After experimental work, it was determined by the applicant that if the free base of compound 3 was used (rather than HCL) as the reaction partner in step J-2, the addition of a base subsequently in the reaction was not necessary. LC-MS determined that the starting material conversion CG689G is >95%. The reaction was more efficient and resulted in higher product content than the previously used mechanism. Importantly, this change also avoided the hydrochloride salt dissociation previously seen step in J-4 (described above).

Ulodesine free form CG6891 using this method was able to be obtained with high HPLC purity and chiral purity (99.1%, 98.6% respectively). Analytical testing confirmed that, by modifying this earlier step, reliable and consistent production of the correct hemi-glutarate salt CG689J from Ulodesine was again possible from a complete method of Ulodesine manufacture.

### Demonstration Batch of Ulodesine Hemi-Glutarate (35 g)

It was then desirable to produce batch amount (35 g) of the glutarate salt product by this new method to demonstrate the potential for use in pharmaceutical processing and biological testing.

To that end, the alternative production route (using a free form of Compound 3 as a reaction partner in the Ulodesine process) was used to make the free base of ulodesine CG6891 as described in the modified process. Thereafter, the newly determined recrystalisation process (herein described above “J”) was utilised to make the final 35 g demonstration batch of the desirable hemi-glutarate salt CG689J.

### Demonstration Batch Data

| Batch No. | Amount | HPLC purity | e.e | Overall Yield |
|---|---|---|---|---|
| CP-0031535-13 | 34.9 g | 99.12% | 98.6% | 8.2% |

### Demonstration Batch 1 (35 g) CP-0031535-13 Analysis:

| Test Items | Analytical Method | Observation |
|---|---|---|
| Appearance | Visual | White crystalline solid powder |
| Identification | ¹H-NMR | Conform to structure |
| Purity | HPLC | 99.12% (a/a) |
| Enantiomeric purity | HPLC | 98.6% |
| Assay | ¹H NMR | 96.0-103.0% (w/w) |
| Residual solvents | GC | EtOH: 0.03%, MeCN: 0.02% |
| Water | K.F. | 0.147% (w/w) |
| Peak temperature | DSC | Complies with peak temperature of |
| | | salt-selection batch |
| Polymorph | XRPD | Complies with XRPD of salt-selection |
| | | batch |

In summary, the desired product Ulodesine hemi-glutarate was obtained in good yield in both batches. The resulting product was verified to be chemically pure and characteristics of the salt was consistent with what was reported in earlier salt selection studies. It is therefore considered that the newly identified and characterised salt of ulodesine and further the new method of production used to make it are highly useful solution for providing ulodesine pharmaceuticals and the treatment of conditions or diseases using the same.

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1. A pharmaceutically acceptable salt of a ulodesine compound: 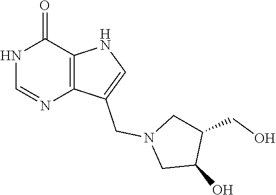 [SMILES: O=c1[nH]cnc2c(CN3C[C@H](CO)[C@@H](O)C3)c[nH]c12]
wherein the salt comprises at least one salt selected from a glutarate, a malonate, and/or an oxalate salt.

2. The salt according to claim 1, wherein the salt comprises a hemi glutarate salt.

3. The salt according to claim 2, wherein the salt comprises a hemi L-glutarate salt.

4. The salt according to claim 1 wherein the salt is a mixed salt and further comprises a hemi succinate salt.

5. A pharmaceutical composition comprising the ulodesine salt compound according to claim 1 and a pharmaceutically acceptable excipient.

6. A method of treating a disease in a subject in need thereof said method comprising administering the ulodesine salt according to claim 1 or a pharmaceutical composition comprising the ulodesine salt of claim 1 to the subject.

7. A method of preparing a ulodesine hemi glutarate salt or a ulodesine hemi L-glutarate salt comprising the steps of:
(a) preparing a solution of ulodesine free base in water, and optionally stirring at room temperature;
(b) adding glutarate acid, or L-glutarate acid to the mixture of step (a), and optionally stirring for 30 min at room temperature;
(c) freeze-drying the solution of step (b) to yield a white solid product;
(d) dissolving the solid product of step (c) in water; optionally heating up to 75° C.; adding ethanol and optionally stirring at 75° C. for 30 minutes, to form a homogenous solution;
(e) making a dropwise addition of acetonitrile to the solution of step(d), optionally over a period of 60 minutes;
(f) stirring the solution of step (e) for 60 minutes, optionally at 75° C.; and optionally cooling the solution to 0° C. over a period of 60 minutes;
(g) filtering and washing with acetonitrile to obtain ulodesine hemi salt glutarate hemisalt.

8. The method of preparing a ulodesine hemi glutarate salt according to claim 7, wherein:
step (a) requires the use of a free form of the reactant: 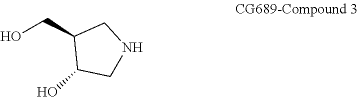
in the preparation of ulodesine freebase in water.

9. The method of claim 6, wherein the disease is selected from gout, skin disorders, cancer, B ant T-cell mediated disease, bacterial infection, and protozoal infection.
